(19) United States
(12) Patent Application Publication
(10) Pub. No.: US 2004/0241734 A1
(43) Pub. Date: Dec. 2, 2004

(54) Methods for in situ hybridization without the need for competitior DNA

(75) Inventor: Lisa Davis, Albuquerque, NM (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Exagen Diagnostics

(21) Appl. No.: 10/833,954

(22) Filed: Apr. 28, 2004

Related U.S. Application Data
(60) Provisional application No. 60/466,552, filed on Apr. 28, 2003.

(51) Int. Cl. C12Q 1/68

(52) U.S. Cl. 435/6

(57) ABSTRACT

The present invention provides novel methods for in situ hybridization in the absence of competitor DNA.

### CROSS REFERENCE

This application claims priority to U.S. Provisional Patent Application Ser. No. 60/466,552 filed Apr. 28, 2004.

### FIELD OF THE INVENTION

This invention relates generally to the hybridization process whereby the pairing of complementary single stranded DNA molecules is accomplished. More particularly the present invention provides methods for conducting in situ hybridization that eliminates the need for competitor DNA.

### BACKGROUND

Chromosome abnormalities are associated with genetic disorders and exposure to agents known to cause degenerative diseases, particularly cancer. Chromosome abnormalities fall into three general types: 1. Extra or missing individual chromosomes, 2. Extra or missing portions of a chromosome, and 3. Chromosomal re-arrangements. Detectable chromosomal abnormalities occur with the frequency of one in every two hundred fifty human births. Abnormalities that involve deletions or additions of chromosomal material alter the gene balance of an organism and generally lead to fetal deaths or to serious mental and/or physical defects.

As a result of these serious consequences, clinical cytogeneticists are constantly on the look out for quicker methods of evaluating the nature of any chromosomal anomalies. One such method is In Situ hybridization (ISH), which also is use to localize and detect specific DNA and mRNA sequences in morphologically preserved tissue sections, cell preparations, or chromosomes by hybridizing the complimentary strand of a nucleic acid probe to the sequence of interest. When the chemical tag is fluorescence, the technique is called fluorescence In Situ hybridization or FISH.

The basis for specific DNA detection in a complex mixture is base pair complementarities between the probe and the target. However, most cloned DNA probes contain both a unique sequence component, specific for the genome region of interest in the target, and repetitive sequence DNA elements that are not specific for the target region of interest. Repetitive sequence DNA elements are a family of abundant ubiquitous DNA sequences comprising closely related, but not completely identical base pair sequences. Most cloned probes used in ISH contain repetitive sequences that are randomly dispersed throughout most genomes. Dispersed repetitive sequences such as Alu-repeats make up a huge part of a human genome. Thus, larger probes made from this genome have an increasing probability of containing such repetitive elements. This renders many cloned ISH probes not entirely unique to the chromosome of interest and will result in various nonspecific hybridization all over the genome.

Hybridization of these repetitive sequences can be disabled in several ways. The most common method for disabling repetitive sequences is to block the repetitive sequence/sequences by pre-association with unlabeled repetitive sequence containing complimentary fragments such as COT-1DNA and generically known as competitor DNA. The repetitive sequences in the probe find their complimentary unlabeled sequences in the competitor DNA and rapidly form double stranded DNA over their entire length or complimentary of the sequence. Since only single stranded probe DNA is able to hybridize to the single stranded target DNA, the repetitive DNA sequence in the probe is no longer available to hybridize to the target.

This method is some times referred to as chromosome In Situ suppression, or suppression of repetitive sequences, and requires preparation or purchase from a commercial source of large quantities of competitor DNA which can be prohibitively time consuming and expensive.

In order to circumvent the use of competitor DNA as a means of eliminating non-specific signal from repetitive DNA elements, techniques have been developed based upon solution hybridization and affinity capture, for physically removing the repetitive sequence elements from a probe prior to its use in the hybridization reaction. However, this approach requires a time consuming and dedicated approach to development of each probe. While this is practical for production of large quantities of individual probes, it does not lend itself as a routine procedure for multiple analyses with large numbers of probes.

Another technique for increasing the specificity of a probe without an excess of competitor DNA requires a pre-annealing step of the probe to itself in order to allow the labeled repetitive sequence elements of the probe to self-anneal prior to hybridization to the target DNA. This step requires an additional manipulation step in the hybridization procedure.

Still another procedure for increasing the specificity of the unique sequence component of a probe containing both unique sequence and repetitive elements is to take advantage of DNA: DNA hybridization kinetics during the post hybridization wash step. The wash step comprises washing the material, post hybridization, at or close to the conditions at which the hybridization takes place, to remove unbound probe or probe material which has loosely bound to imperfectly matched sequences. In this approach the DNA repeat element component of the probe is selectively de-natured after hybridization to the target, by virtue of a higher instability of repeat element sequence mismatches, relative to perfect based pair matches of the unique sequence component. This repetitive sequence component of the probe is thus removed from the target during the washing step. This procedure is referred to as differential destabilization of repetitive sequence hybrids.

Typically, probes supplied by commercial entities are provided with competitor DNA in the “probe mix.” Thus, as commercially available, the hybridization probe solutions have competitor DNA present.

Therefore, it would be useful to provide a method for increasing specificity of probes for targets in the absence of competitor DNA. This would lead to more rapid hybridization and decreased costs associated with in situ hybridization.

### SUMMARY OF THE INVENTION

The present invention provides methods for in situ hybridization comprising

(a) contacting the biological specimen on a platform with a (i) hybridization solution; and (ii) one or more labeled probes;

(b) hybridizing the one or more labeled probes to one or more target nucleic acid sequences in the biological specimen;

(c) removing unbound labeled probe from the biological specimen; and

(d) detecting labeled probe that has hybridized to the one or more target nucleic acid sequences;

wherein each of steps (a)-(d) is conducted in the absence of competitor DNA.

### DETAILED DESCRIPTION OF THE INVENTION

All references cited are herein incorporated by reference in their entirety.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

In one aspect, the present invention provides methods for in situ hybridization comprising

(a) contacting a biological specimen on a platform with a (i) hybridization solution; and (ii) one or more labeled probes;

(b) hybridizing the one or more labeled probes to one or more target nucleic acid sequences in the biological specimen;

(c) removing unbound labeled probe from the biological specimen; and

(d) detecting labeled probe that has hybridized to the one or more target nucleic acid sequences;

wherein each of steps (a)-(d) is conducted in the absence of competitor DNA.

As used herein, the term “biological specimen” refers to any specimen for which ISH is useful, including but not limited to fixed cell and tissue samples including but not limited to blood smears, surgical specimens, pathology specimens, and bone marrow cells; and chromosomal spreads. Such a biological specimen is typically fixed either on the platform or prior to placement on the platform. As used herein, the term “fixed” refers to use of a reagent that preserves cell and tissue constituents in as close a life-like state as possible and to allows them to undergo further analytic procedures without change. Fixation also arrests autolysis and bacterial decomposition and stabilizes the cellular and tissue constituents so that they withstand the subsequent stages of tissue processing. The selection of an appropriate fixative is based on considerations such as the structures and entities to be demonstrated and the effects of short-term and long-term storage. Each fixative has advantages and disadvantages. Some are restrictive while others are multipurpose. In non-limiting examples, the fixative can be one or more of a buffered formalin solution, aldehydes, such as formaldehyde and, glutaraldehyde; oxidizing agents such as metallic ions and complexes, such as osmium tetroxide, chromic acid; protein-denaturing agents, such as acetic acid, methyl alcohol (methanol), and ethyl alcohol(ethanol); mercuric chloride; picric acid; microwaving; excluded volume fixation; and vapour fixation. Such fixatives are widely available as is known to those of skill in the art. Chromosomal spreads can be prepared by any method known to those of skill in the art.

As used herein, the term “platform” refers to any substrate upon which ISH can be performed, including but not limited to glass or plastic slides, tissue culture plates, and multi-well tissue culture plates. As will be apparent to those of skill in the art, the platform can contain a single biological specimen, or may contain multiple biological specimens, such as in a tissue microarray.

The target nucleic acid sequence is any one or more sequences present in the biological specimen that are to be detected, and can comprise DNA or RNA, and may be derived from nuclear, mitochondrial, or pathogenic/infectious sources.

As used herein, the term “labeled probe” refers to a single stranded or double stranded nucleic acid sequence, preferably a double stranded DNA sequence, with a detectable moiety attached (either prior to the hybridization and removal of unbound probe steps, or subsequent to these steps and prior to the detection step). It is most preferred that the labeled probe comprises a cloned DNA that contains repetitive sequence elements. When more than one labeled probe is used, it is preferred that the detectable labels on the different probes are distinguishable from each other, for example, to facilitate differential determination of their signals. Methods for detecting the label include, but are not limited to spectroscopic, photochemical, biochemical, immunochemical, physical or chemical techniques. For example, useful labels include but are not limited to radioactive labels such as ³²P, ³H, and ¹⁴C; fluorescent dyes such as fluorescein isothiocyanate (FITC), rhodamine, lanthanide phosphors, and Texas red, ALEXIS™ (Abbott Labs), CY™ dyes (Amersham); electron-dense reagents such as gold; enzymes such as horseradish peroxidase, beta-galactosidase, luciferase, and alkaline phosphatase; colorimetric labels such as colloidal gold; magnetic labels such as those sold under the mark DYNABEADS™; biotin; dioxigenin; or haptens and proteins for which antisera or monoclonal antibodies are available. The label can be directly incorporated into the polynucleotide, or it can be attached to a probe or antibody which hybridizes or binds to the polynucleotide. The labels may be coupled to the probes by any means known to those of skill in the art. In a various embodiments, the probes are labeled by chemical coupling of a detectable label to the probe, nick translation, PCR, or random primer extension (see, e.g., Sambrook et al. supra).

As used herein “contacting” includes the step of denaturing the target nucleic acids in the biological specimen and the probe. While this process can take place prior to addition of the hybridization solution (for example, in a separate denaturing solution), it is preferred that denaturation of the target nucleic acid and probe, as well as hybridization, occur in the same solution. While the contacting of the biological specimen with the hybridization solution can occur simultaneously with or prior to contacting of the biological specimen with the labeled probe, it is preferred that the labeled probes are added to the hybridization solution and that denaturation of the target nucleic acid and the probes occurs simultaneously in the hybridization solution.

Such denaturation can be accomplished by any means known in the art. In a preferred embodiment, the labeled probes and nucleic acid targets are simultaneously denatured for approximately 1.5.+−0.0.5 minutes in an oven of approximately 100° C.+−5° C., and then placed in appropriate hybridization conditions as discussed below.

Any conditions in which the labeled probe binds selectively to the target nucleic acid sequence to form a hybridization complex, and minimally or not at all to other sequences, can be used in the methods of the present invention. The exact conditions used will depend on the length of the polynucleotides probes employed, their GC content, as well as various other factors as is well known to those of skill in the art. (See, for example, Tijssen (1993) Laboratory Techniques in Biochemistry and Molecular Biology-Hybridization with Nucleic Acid Probes part I, chapt 2, “Overview of principles of hybridization and the strategy of nucleic acid probe assays,” Elsevier, N.Y. (“Tijssen”)). In one embodiment, stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent wash conditions is a 0.2×SSC wash at 65° C. for 15 minutes (see, e.g., Sambrook (1989) Molecular Cloning: A Laboratory Manual (2nd ed.) Vol. 1-3, Cold Spring Harbor Laboratory, Cold Spring Harbor Press, NY (“Sambrook”) for a description of SSC buffer). Often, a high stringency wash is preceded by a low stringency wash to remove background probe signal.

In a preferred embodiment, the methods utilize hybridization buffers disclosed in U.S. Pat. Nos. 5,750,340 and 6,022,689, incorporated by reference herein in their entirety. In a more preferred embodiment, one of hybridization buffers F or G is used, as disclosed in U.S. Pat. No. 5,750,340:

F: 10%+/−2% by weight dextran sulfate

10%-30% (preferably 20%) by volume formamide

0.9% by weight salt (NaCl, KCl, or other appropriate salt)

G: 10%+/−2% by weigh dextran sulfate

15-25% glycerol (preferably 20%)

0.9% by weight salt (NaCl, KCl, or other appropriate salt)

The use of these hybridization buffers decreases the number of steps required for ISH. For example, other methods involve various laborious steps, separate denaturation of target nucleic acids and labeled probes, separate denaturation and hybridization procedures, and repeated dehydration of target nucleic acids with graded alcohols. Use of the preferred hybridization buffers simplifies the required steps and decreases the time required to carry out the ISH.

In a preferred embodiment, hybridization solution G is used. While not being bound by a specific mechanism, it is believed that the greater viscosity of glycerol compared to formamide facilitates denaturation of mismatches between repetitive sequences in the probe and the target nucleic acid, and thus is ideally suited for use without competitor DNA.

Any wash conditions can be used that minimize the retention of unbound probe to the bodily fluid sample on the solid support. As will be appreciated by those of skill in the art, this step does not require that all unbound probe is removed, but simply that enough unbound probe is removed to permit adequate detection of the bound probe to the target nucleic acid.

In a preferred embodiment, unbound probe is removed by washing with 50% formamide in 0.45% NaCl for 3 minutes at 38° C., and then for 5 minutes in 0.9% NaCl at 38° C. (for use with formamide-containing hybridization solutions). Alternatively, the hybridized slides are preferably washed in formamide-free 0.1-0.2% NaCl at between 55 and 65° C. for 2-10 minutes; preferably at 60° C. for 5 minutes and then for another 1-5 minutes, preferably 3 minutes in fresh 0.1-0.2% NaCl at between 55 and 65°, preferably at 60° C. In a preferred embodiment, the formamide-free wash conditions are used.

Any desirable post-hybridization processing steps can be carried out. It is preferred that the biological specimens on the platform are dried, such as by air-drying, prior to detection. Optionally, the biological samples can be counterstained to detect cell structures, such as counterstaining with 4,6-diamidino-2-phenylindole (DAPI) or propidium iodide (PI) solution to stain nuclei.

Signals from the labeled probes can be detected by any means known in the art for the particular label employed. For example, where the detectable label is a fluorescent label, one can detect fluorescence signals by visualization with a fluorescence microscope.

### EXAMPLES

Probes:

Flourescent probes were prepared from BACs (bacterial artificial chromosomes) (obtained from CHORI, Children's Hospital Oakland research Institute) by labeling with Spectrum Orange or Spectrum Green (Abbott Laboratories) using the Bio-Prime labeling kit (Invitrogen, Inc.) according to manufacturer's protocol. The probes were diluted into the denaturation-hybridization solution (F or) G at final concentrations of 40 ug/ml (400 ng probe in 10 uL solution per slide).

Denaturation-Hybridization Solutions:

Solution G: Formamide-free denaturation-hybridization solution: 10% dextran sulfate, 20% glycerol, and 0.9% NaCl. To prepare: Dissolve 1 gram of dextran sulfate and 0.09 gram of NaCl in 8 ml deionized water. Add 2 ml of glycerol. Thoroughly mix and store at −20° C. until use.

Solution F: Formamide-containing denaturation hybridization solution: 10% dextran sulfate, 20% formamide, and 0.9% NaCl. To prepare: Dissolve 1 gram dextran sulfate and 0.09 grams NaCl at −20° C. until use.

Post-Hybridization Wash Solutions:

Formamide-containing washings: The hybridized slides were immersed in 50% formamide solution containing 0.45% NaCl for 3 minutes at 38° C., and subsequently in 0.9% NaCl for 5 minutes.

Formamide-free washings: The hybridized slides were immersed in. 0.1%-0.2% NaCl at 60° C. for 5 minutes and then for another 3 minutes at 60° C. in new 0.1%-0.2% NaCl solution.

Cells and tissues. Metaphase and interphase chromosome preparations were examined. Metaphase chromosomes were prepared from peripheral blood and bone marrow cells. Interphase nuclei were prepared from formalin fixed, paraffin-embedded tumor and biopsy samples.

PBMC Metaphase chromosomes. Peripheral blood culture was performed according to common protocols, by inoculating sodium heparin collected whole blood in 1640 RPMI/10% FCS and PHA stimulation. 72 hours cultures were subjected to colchicine (Colcemid, Life Technologies, Gaithersburg, Md., USA) for 40 minutes, and the cells were then resuspended in 0.075M KCL hypotonic buffer for 15 minutes at 37° C. A small volume of 3:1 methanol:acetic acid fixative was added to the cell suspension and 3 more fixative washes were subsequently performed. Cell pellet(s) were stored at −20° C. in fixative until ready for FISH.

Fixed cells were dropped onto on dry, commercially pre-cleaned microscope slides. Ten ul of fixed cells were placed on microscope slide and the fixative allowed to evaporate. After the slide was visibly dry, 10 uL of the probe mixture (in solution G) was applied, and the slide was heated to 100° C. for 1.5 minutes. The hybridization occurred during the 30 minute, controlled temperature descent to 30° C. Specifically, the temperature cycled through six successive 10-degree temperature extremes ten times each. That is, the temperature was controlled to fluctuate between 90° C. and 80° C. ° 10×, then between 80° C. and 70° C. 10×, 70° C. ° and 60° C. 10×, etc., until the slide reached 30° C.

Post-hybridization Wash. The cover glasses were removed and excess probe was washed at 60° C. for 5 minutes and then for another 3 minutes at 60° C. 15 ul of a mixture of antifade (Vector Laboratories) and counterstain (DAPI or PI) were placed on each sample and cover glassed. The slide was viewed with a Texas red triple bandpass filter (Texas Red and FITC) on an fluorescence microscope; signals were visualized with a 40× dry objective lens in the interphase nuclei.

Bone Marrow Cells. The bone marrow cell fixation procedure for metaphase cytogenetic preps was the same as the PBMC, except that the cells were not cultured prior to fixation in 3:1 methanol acetic acid. Cells that had been fixed and frozen at −20° C. in the fixative for several months after fixation were used in these examples.

Simultaneous Denaturation and Hybridization.

Denaturation. Fixed cells were dropped onto on dry, commercially pre-cleaned microscope slides. Ten ul of fixed cells were placed on microscope slide and the fixative allowed to evaporate. After the slide was visibly dry, 10 uL of the probe mixture (in solution G) was applied, and the slide was heated to 95° C. for 2 minutes. A glass coverslip was gently applied to cover the probe solution and slight pressure applied to assure uniform spread of the probe solution over the sample area. Sealing between the coverslip and glass slide with rubber cement was not necessary. The slides were put into an oven of 95° C. for 2 minutes for denaturation. During this period, both target nucleic acids and labeled probes appeared to be simultaneously and effectively denatured in the presence of the solution G.

Hybridization. Following the 2 minute denaturation, denaturation, the oven was re-set to 55° C. and the temperature allowed to steadily drop over a period of 10 minutes. After an additional 30 minutes at 55° C., the slides were washed.

Post-hybridization Wash. The cover glasses were removed and excess probe was washed in 0.1%-0.2% NaCl for 5 minutes and then for another 3 minutes in new 0.1%-0.2% NaCl solution. 15 ul of a mixture of antifade (Vector Laboratories) and counterstain (DAPI or PI) were placed on each sample and cover glassed. The slide was viewed with a Texas red triple bandpass filter (Texas Red and FITC) on an fluorescence microscope; signals were visualized with a 40× dry objective lens in the interphase nuclei.

Interphase nuclei were prepared from formalin fixed paraffin embedded (FFPE) tissues. The tissues were fixed and embedded using standard histological procedures for surgical specimens. After sections were attached to glass microscope slides, the tissues were deparaffinized using Citri-Solv (Fisher Scientific) (three changes of of solution, 10 minutes each at room temperature), then 2 washes in 100% ethanol at room temperature for 5 minutes. The slides were then air dried, followed by a 20 minute wash in 0.2 N HCL at room temperature, followed by a water rinse and 2 2×SSC washes at room temperature. The slides were then treated with “Pretreatment reagent” (Abbot Labs, IL) for 30 minutes at 80° C., followed by a water rinse and 2 2×SSC washes at room temperature. The slides were then treated with 0.5 mg/ml proteinase K at 37° C. for 30 minutes, rinsed twice with 2×SSC, air dried and then treated with 10% buffered formalin (10 minutes) and two more rinses with 2×SSC. The slides were then air dried.

Simultaneous Denaturation of Samples and Probes

The labeled probes were diluted with solution F or G to an appropriate concentration. Thus, the “probe solution” represents labeled probes which were diluted in solutions F or G. In this example, solution G was used.

Ten uL of the diluted probe solution were spotted on tissue sections on the glass slides. A glass coverslip was gently applied to cover the probe solution and slight pressure applied to assure uniform spread of the probe solution over the sample area. The coverslip and glass slide were sealed with rubber cement for this example. The slides were put into an oven of 95° C. and denatured for 2 minute. During this period, both target nucleic acids and labeled probes appeared to be simultaneously and effectively denatured.

Hybridization

Following denaturation, the slides were transferred into another oven of 55° C. and hybridized overnight.

Post-Hybridization Washings

After hybridization, the rubber cement was removed and the coverslips were removed from the glass slides. The hybridized slides were immersed in. 0.1%-0.2% NaCl for 5 minutes and then for another 3 minutes in new 0.1%-0.2% NaCl solution. 15 ul of a mixture of antifade (Vector Laboratories) and counterstain (DAPI or PI) were placed on each sample and cover glassed. The slide was viewed with a Texas red triple bandpass filter on an fluorescence microscope; signals were visualized with a 40× dry objective lens in the interphase nuclei.

I claim:

1. A method for in situ hybridization comprising
(a) contacting the biological specimen on a platform with
(i) a hybridization solution comprising a solution selected from the group consisting of (A) 10%+/−2% by weight dextran sulfate; 10%-30% by volume formamide; and 0.9% by weight salt; and (B) and 10%+/−2% by weight dextran sulfate; 15-25% glycerol; and 0.9% by weight salt; and
(ii) one or more labeled probes;
(b) hybridizing the one or more labeled probes to one or more target nucleic acid sequences in the biological specimen;
(c) removing unbound labeled probe from the biological specimen; and
(d) detecting labeled probe that has hybridized to the one or more target nucleic acid sequences;
wherein each of steps (a)-(d) is conducted in the absence of competitor DNA.

2. The method of claim 1 wherein the hybridization solution 10%+/−2% by weight dextran sulfate; 15-25% glycerol; and 0.9% by weight salt
